(19) United States
(12) Patent Application Publication
(10) Pub. No.: US 2015/0320949 A1
(43) Pub. Date: Nov. 12, 2015

(54) RESPIRATORY MEASUREMENT APPARATUS HAVING INTEGRATED FILTER

(71) Applicant: KONINKLIJKE PHILIPS N.V., EINDHOVEN (NL)

(72) Inventor: Michael Brian JAFFE, CHESHIRE, CT (US)

(21) Appl. No.: 14/367,250

(22) PCT Filed: Dec. 21, 2012
(86) PCT No.: PCT/IB2012/057581

§ 371 Date: Jun. 20, 2014

Related U.S. Application Data
(60) Provisional application No. 61/580,344, filed on Dec. 27, 2011.

(51) Int. Cl. A61M 16/00; A61M 16/16; A61M 16/08; A61M 16/10; A61B 5/087

(52) U.S. Cl. CPC A61M 16/0003; A61M 16/1055; A61B 5/087; A61M 16/0866; A61M 16/0858; A61M 16/16; A61M 2016/0027; A61M 2016/0036

(57) ABSTRACT

An apparatus comprises a flow sensor configured to sense airflow between a respiration machine and a patient, a first connector configured to communicate air between the flow sensor and the patient, a second connector configured to communicate air between the flow sensor and the respiration machine, multiple pressure sensing ports configured for connection to pressure sensing tubes and configured to communicate gas pressure between the flow sensor and a pressure flowmeter, and a filter integrated with the flow sensor between the first connector and the pressure sensing ports and configured to communicate gas pressure therethrough while preventing contaminants from passing from the flow sensor to the pressure sensing tubes.

### TECHNICAL FIELD

The present invention relates generally to technologies for respiratory flow and/or respiratory gas measurement. More particularly, various inventive systems and apparatuses disclosed herein relate to ventilator or respirator systems having a flow and/or gas sensor with an integrated filter designed to prevent cross-contamination between different patients.

### BACKGROUND

Respiratory flow and gas measurements are commonly performed in ventilator and respirator systems. Such measurements can be used, for instance, to regulate the supply of breathable air to a patient in an intensive care unit (ICU), or to monitor the breathing patterns of a person receiving therapy.

Certain technologies for respiratory measurements are governed by standard setting organizations to ensure quality and safety. For example, the International Electrochemical Commission (IEC) defines standards for critical care ventilators used in ICUs, as well as components connected to the ventilators.

Among IEC's standards, there are requirements that critical care ventilators and their accessories prevent cross-contamination between different ICU patients. One such requirement is provided by IEC 60601-2-12, which states “Gas pathways through the VENTILATOR and its ACCESSORIES that can become contaminated with body fluids or expired gases during NORMAL CONDITION or SINGLE FAULT CONDITION shall be designed to allow dismantling for cleaning and disinfection or cleaning and sterilization.”

Unfortunately, some of these requirements may be cumbersome and inefficient to implement. For example, in a typical ICU environment, dismantling and sterilization procedures can be time consuming, inconvenient, and potentially expensive. Accordingly, it is desirable to design ventilator and respirator systems and their accessories that avoid contamination in order to obviate the need for such procedures.

### SUMMARY

The present disclosure is directed to inventive systems and apparatuses for performing respiratory flow and/or respiratory gas measurements. More specifically, the disclosed systems and apparatuses include a flow sensor with an integrated filter designed to prevent cross-contamination between different users, such as different ICU patients. For example, in some embodiments a ventilator or respirator system comprises a pressure or flow sensor having a housing connected to two pressure sensing tubes. The pressure sensing tubes are connected to a pressure flowmeter to quantify airflow through the flow sensor. A filter, such as a bacterial or viral filter, is integrated into the housing of the flow sensor to prevent contamination from being communicated from a patient to the pressure sensing tubes. The filter is generally integrated with a portion of the housing adjacent to the sensing tubes or a portion adjacent to a patient-side connector.

Because the integrated filter prevents contamination from entering the pressure sensing tubes, it substantially eliminates the problem of cross-contamination that may otherwise occur through the pressure sensing tubes or the pressure flowmeter. This in turn eliminates a need to disassemble and sterilize the pressure sensing tubes or the pressure flowmeter to address such contamination.

Generally, in one aspect, an arrangement is provided for measurement of respiratory flow or respiratory gases in an airway between a patient and a ventilator or respirator. The arrangement comprises an adapter comprising a housing having first through fourth openings, a first connector attached to the adapter at the first opening and configured for connection to a first hose leading toward the ventilator or respirator, a second connector attached to the adapter at the second opening and configured for connection to a second hose leading toward the patient, third and fourth connectors attached to the adapter at the third and fourth openings and configured for connection with pressure sensing tubes to be used in conjunction with a differential pressure flow sensor located along a path between the first and second connectors, and a filter integrated with the housing of the adapter and configured to prevent patient originated infective agents from contaminating the pressure sensing tubes.

In some embodiments, the filter comprises a filter housing molded to the housing of the adapter. In some embodiments the arrangement further comprises a pressure flowmeter connected to the pressure sensing tubes and configured to measure airflow through the adapter based on a pressure differential across the pressure sensing tubes. In some embodiments, the filter is located adjacent to the third and fourth connectors. In some embodiments, the filter is located adjacent to the first connector. In some embodiments, the adapter further comprises a resistive element adapted to create a pressure differential between the pressure sensing tubes.

In another aspect, a system comprises a patient circuit configured to be connected between a ventilator and a patient, a differential pressure sensor disposed in-line with the patient circuit and the patient and configured to sense a respiratory gas flow of the patient, the differential pressure sensor comprising a housing and first and second pressure sensing ports connected to the housing, and a flowmeter comprising first and second input ports in communication with the first and second pressure sensing ports of the differential pressure sensor, wherein the flowmeter is configured to measure a differential pressure between the first and second input ports and to output an electrical signal responsive to the differential pressure. The system further comprises first and second pressure sensing tubes connected between the first and second pressure sensing ports of the differential pressure sensor and the first and second ports of the flowmeter, and a contaminant blocking device integrated with the housing of the differential pressure sensor and configured to prevent contamination from being transmitted between the patient and the pressure sensing tubes.

In some embodiments, the patient circuit comprises a dual-limb patient circuit including a wye element having first and second ports connected to the dual limbs and having a third port connected to the differential pressure sensor. In some embodiments, the contaminant blocking device comprises a filter housing connected to the housing of the differential pressure sensor, and a filter element mounted in the filter housing. In some embodiments, the filter housing is molded to the housing of the differential pressure sensor. In some embodiments, the filter housing is bonded to the housing of the differential pressure sensor. In some embodiments, the contaminant blocking device is disposed adjacent to the pressure sensing tubes. In some embodiments, the contaminant blocking device is disposed in-line with an airflow passing through the differential pressure sensor.

In another aspect, an apparatus comprises a flow sensor configured to sense airflow between a respiration machine and a patient, a first connector configured to communicate air between the flow sensor and the patient, a second connector configured to communicate air between the flow sensor and the respiration machine, multiple pressure sensing ports configured for connection to pressure sensing tubes and configured to communicate gas pressure between the flow sensor and a pressure flowmeter, and a filter integrated with the flow sensor between the first connector and the pressure sensing ports and configured to communicate gas pressure therethrough while preventing contaminants from passing from the flow sensor to the pressure sensing tubes.

In some embodiments, the filter is integrally formed with a housing of the flow sensor. In some embodiments, the filter comprises filter housing molded to a wall of the flow sensor. In some embodiments, the pressure sensing ports are located in the filter housing. In some embodiments, the filter is a pleated bacterial filter. In some embodiments, the respiration machine is a medical ventilator configured for use in an intensive care unit.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a functional block diagram of a ventilator comprising an in-line proximal differential pressure based flow sensor with pressure sensing tubing according to a representative embodiment.

FIG. 2 is a detailed illustration of a portion of the ventilator system of FIG. 1 according to a representative embodiment.

FIGS. 3A and 3B are cross-sectional diagrams of an in-line proximal differential pressure sensor according to a representative embodiment.

FIG. 4 is a functional block diagram of a ventilator system comprising an in-line proximal differential pressure based flow sensor with an integrated filter according to a representative embodiment.

FIG. 5 is a functional block diagram of another ventilator system comprising an in-line proximal differential pressure based flow sensor with an integrated filter according to a representative embodiment.

FIG. 6 is a conceptual block diagram illustrating an adaptor for a ventilator or respirator system according to a representative embodiment.

### DETAILED DESCRIPTION

As discussed above, cross-contamination can be a substantial problem in equipment used for respiratory flow and/or gas measurement. For example, in a typical ventilator system, contagions can be communicated from one patient to another by contaminating pressure sensing tubes connected to a flow sensor in the case of an undetected SINGLE FAULT CONDITION. Moreover, conventional approaches for preventing cross-contamination may require the equipment to be disassembled and sterilized between successive uses, which can be time consuming, expensive, and inconvenient in many operational settings.

The inventors have therefore recognized and appreciated that it would be beneficial to provide systems and apparatuses for respiratory flow and/or gas measurement that prevent contagions or other contaminants from entering pressure sensing tubes through the housing of a flow and/or gas sensor. Accordingly, various embodiments are directed to systems and apparatuses in which a filter is integrated into a flow and/or gas sensor to prevent contaminants from entering pressure sensing tubes attached to the housing.

The described embodiments are particularly relevant to respiration equipment used in medical environments such as ICUs. For example, they can be readily applied to medical ventilator or respirator systems. Nevertheless, the embodiments are not limited to medical applications or equipment, and they can be adapted for use in other settings, such as sports-related respiration equipment.

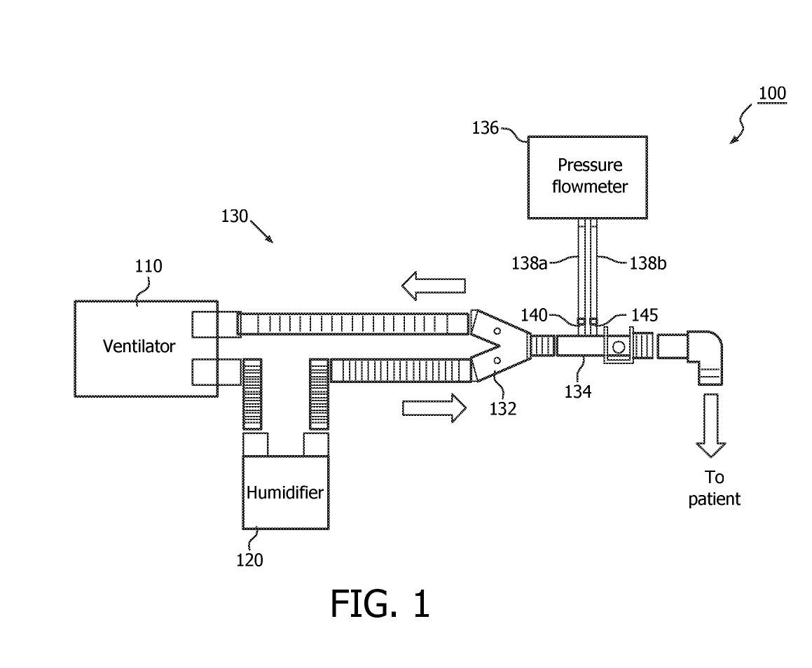

FIG. 1 is a functional block diagram of a ventilator system 100 comprising an in-line proximal differential pressure based flow sensor with pressure sensing tubing according to a representative embodiment.

Referring to FIG. 1, ventilator system 100 comprises a ventilator 110, a humidifier 120, and a patient circuit 130. Patient circuit 130 comprises a wye 132, a pressure sensor 134, a flowmeter 136, and first and second pressure sensing tubes 138a and 138b.

Patient circuit 130 is a dual limb circuit having a first limb connected to ventilator 110 and a second limb connected to ventilator 110 via humidifier 120. It receives expired air from the patient through the first limb, as indicated by a first large arrow pointing toward ventilator 110, and it sends inspired air to the patient through the second limb, as indicated by a second large arrow pointing away from ventilator 110.

Pressure sensor 134, which can also be referred to as a flow sensor, is a differential pressure sensor. It comprises a cylindrical housing that allows air to pass to and from the patient, and two pressure sensing ports 140 and 145 connected to first and second pressure sensing tubes 138a and 138b. Pressure sensor 134 further comprises a resistive element or obstruction located along the inside of the housing between pressure sensing ports 140 and 145. The resistive element changes the speed of airflow as it passes through the housing, which creates a pressure differential between pressure sensing ports 140 and 145. This pressure differential is detected by pressure flowmeter 136 to measure respiratory flow through the housing.

Pressure sensing tubes 138a and 138b typically comprise flexible tubes, which can be the same as any standard tubing. During normal operation, pressure sensing tubes 138a and 138b may be filled with a gas volume or column whose pressure changes in response to respiratory action by ventilator 110 and the patient. These changes in pressure are measured by flowmeter 136. In other words, gas does not normally flow through first and second pressure sensing tubes 138a and 138b from pressure sensor 134 to flowmeter 136.

Although FIG. 1 shows ventilator system 100 with a dual-limb patient circuit and other specific features, the embodiments are not limited to this configuration. For example, in other embodiments a ventilation system can have a single limb patient circuit. In addition, certain concepts described in relation to ventilator system 100 and other embodiments can be applied in alternative types of breathing systems, such as respirator systems. Moreover, the combination of pressure sensor 134 and pressure flowmeter 136 can be further combined with a gas sensor such as CO₂/O₂ sensor. For example, such a sensor could be integrated with the housing of pressure sensor 134 in order to detect the gas composition within.

As shown in FIG. 1, pressure sensor 134 is connected proximal to a patient between the patient and wye 132. In practice, ventilator 110 and humidifier 120 may be installed in a facility such as an ICU, and when a patient is to be ventilated, patient circuit 130, including wye 132, pressure sensor 134 and flowmeter 136 may be separately provided.

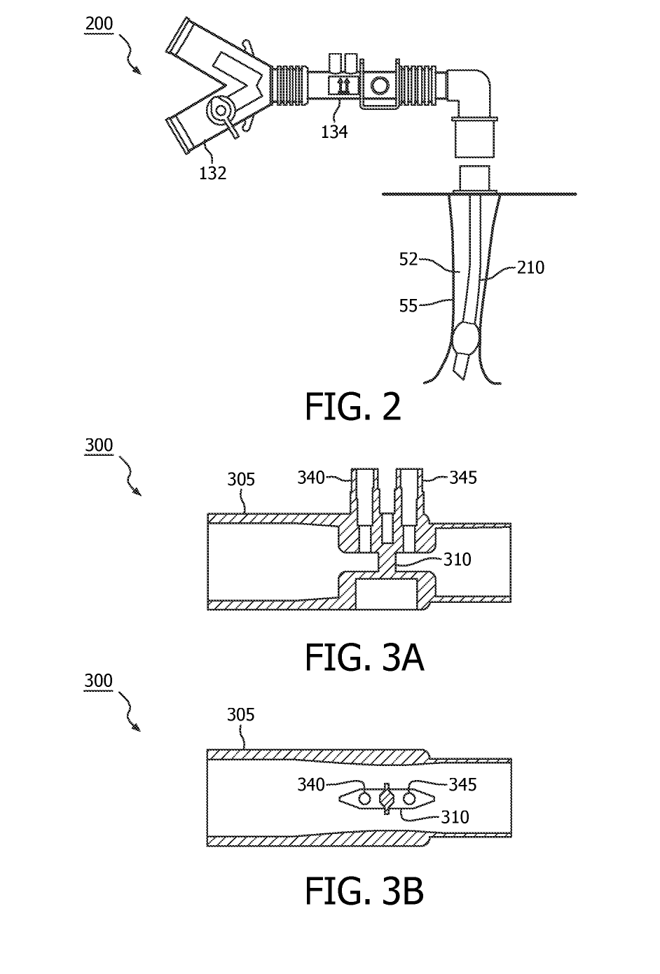

FIG. 2 is a detailed illustration of a portion of ventilator system 100 according to a representative embodiment. For convenience, this portion of ventilator system 100 is labeled as portion 200.

Referring to FIG. 2, pressure sensor 134 is connected to an endotracheal tube 210 inserted into the interior 52 of a patient's trachea 55. Operationally, ventilator 110 supplies gas from a ventilator inspiratory port to humidifier 120. The gas typically comprises room air or an elevated level of oxygen. The gas is generally dry and at room temperature which is nominally 25° C. Gas exiting humidifier 120 is typically at 100% relative humidity (RH) (i.e. saturated) and at a temperature greater than room temperature and less than or equal to body temperature of 37° C. This gas is supplied to the patient via the “lower limb” (“inspired limb”) of patient circuit 130, including wye 132 and pressure sensor 134. Gas returning from the patient is less than 100% RH due to condensation and at a lower temperature (such as 33° C.) and returns to ventilator 110 via the “upper limb” (“expired limb”) of patient circuit 130, including wye 132 and pressure sensor 134.

Pressure sensor 134 operates with flowmeter 136 to measure respiratory gas flow of the patient. Pressure sensor 134 senses gas pressure differentially through the use of a restrictive element, as described above, and first and second pressure sensing tubes 138a and 138b communicate the sensed gas pressure to pressure flowmeter 136. Pressure flowmeter 136 then measures the differential pressure to generate one or more corresponding electrical signals. For example, pressure flowmeter 136 may comprise a diaphragm between two input ports connected to first and second pressure sensing tubes 138a and 138b. The diaphragm may be displaced in response to a pressure differential between the tubes and the displacement may be converted into an electrical signal indicating the direction and/or magnitude of respiratory flow through the housing of pressure sensor 134.

FIGS. 3A and 3B are cross-sectional diagrams of a pressure sensor 300 according to a representative embodiment. In particular, FIG. 3A is a cross-sectional side view of pressure sensor 300, and FIG. 3B is a cross-sectional top view of pressure sensor 300.

Pressure sensor 300 is an in-line proximal differential pressure sensor, and it represents one embodiment of pressure sensor 134. Accordingly, it can be incorporated in a ventilator system such as that illustrated in FIG. 1. Further details of example embodiments of an in-line proximal differential pressure sensor and a pressure flowmeter that can be incorporated in ventilator system 100 may be found in U.S. Pat. No. 5,535,633, the disclosure of which is hereby incorporated by reference.

Referring to FIGS. 3A and 3B, pressure sensor 300 comprises a housing 305, a resistive element 310, and pressure sensing ports 340 and 345. As air passes through housing 305, resistive element 310 restricts flow and creates a pressure differential between pressure sensing ports 340. This pressure differential can be communicated, via pressure sensing tubes, to a pressure flowmeter such as that illustrated in FIG. 1.

For example, during normal operations of ventilator system 100, where there is no leak or fault in patient circuit 130, each of the first and second pressure sensing tubes 138a and 138b is filled with a gas volume or column whose pressure changes in response to respiratory action by ventilator 110 and the patient. The changes in pressure are measured by flowmeter 136. In other words, gas does not normally flow through first and second pressure sensing tubes 138a and 138b from pressure sensor 134 to flowmeter 136.

However, in the event of a single fault condition, it is possible for pressure sensing tubes 138a and/or 138b to become contaminated, for example with body fluids (e.g., liquid matter) from a patient via patient circuit 130, and this contamination could be communicated through pressure sensing tubes 138a and/or 138b to flowmeter 136 if undetected in pressure sensing tubes 138a and/or 138b. In that case, it may be necessary to dismantle, clean, and disinfect and/or sterilize flowmeter 136, which is undesirable.

Accordingly, to address this problem, the inventors have conceived of systems and apparatuses in which a filter is integrated with the housing of a pressure sensor or flow sensor. Such a filter can be, for example, a bacterial and/or viral filter. The filter can be formed of various alternative materials, such as pleated or non-pleated fabrics, for example. Moreover, the filter can be integrated with the housing in various alternative ways. For example, the filter can have a housing that is directly molded to the flow sensor housing, or it can be attached within a cavity formed in the flow sensor housing.

The integrated filter can prevent body fluids or other contaminants, such as gas-borne particles, from reaching a flowmeter or pressure sensing tubes connected to the flow sensor. Moreover, the filter can potentially have other beneficial performance characteristics, such as communicating gas pressure or gas pressure changes without significant attenuation, providing an effective barrier with high gas-borne bacterial removal efficiency, providing use with medical gases such as CO₂, N₂ and O₂, and having a convenient form factor due to integration with the flow sensor. In addition, the integrated filter can be used in a flow sensor that is combined with other functional components, such as a CO₂/O₂ sensor.

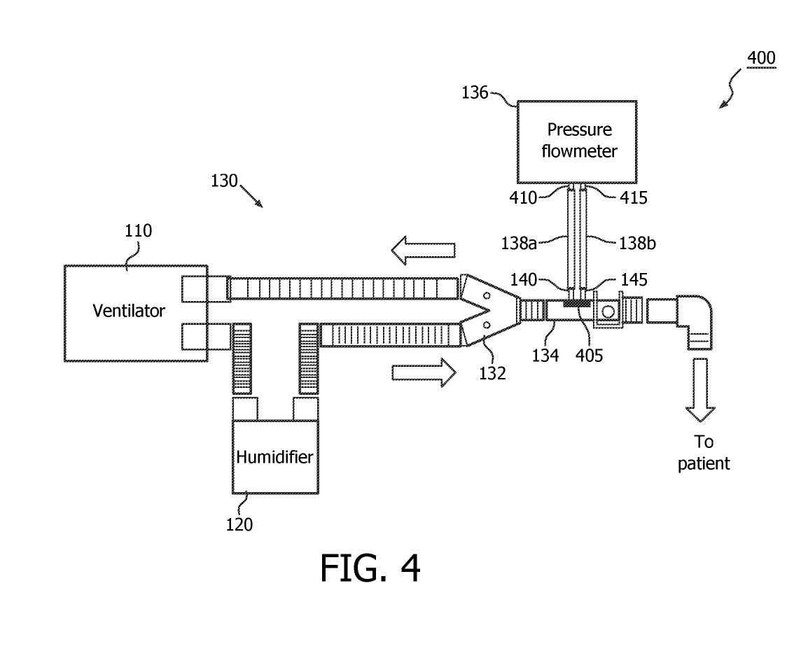

FIG. 4 is a functional block diagram of a ventilator system 400 comprising an in-line proximal differential pressure based flow sensor with pressure sensing tubing and contaminant blocking by an integrated filter. Like elements in ventilator system 400 and ventilator system 100 have the same reference numerals, and a description thereof will not be repeated. Ventilator system 400 is the same as ventilator system 100 described above, except that a filter 405 has been integrated in pressure sensor 134 adjacent to pressure sensing ports 140 and 145. Again, it should be noted that although for illustration of a concrete example FIG. 4 shows a ventilator system 400 having a dual-limb patient circuit, in other embodiments a ventilation system may have a single limb patient circuit.

As seen in FIG. 4, differential pressure sensor 134 comprises first and second pressure sensing ports 140 and 145 having associated first and second connectors, and flowmeter 136 comprises first and second input ports 410 and 415 having associated first and second connectors. Filter 405 has an inlet facing the inside of pressure sensor 134 and an outlet facing pressure sensing tubes 138a and 138b connected to flowmeter 136. Filter 405 is configured to communicate a gas pressure or gas pressure change from differential pressure sensor 134 to flowmeter 136 for pressure measurement, and to prevent contaminants, including for example liquid and airborne particles, from flowing therethrough from differential pressure sensor 134 to flowmeter 136.

Filter 405 can prevent substantially all such contaminants from reaching flowmeter 136, thus eliminating the need for dismantling and cleaning or sterilization of flowmeter 136 when it is deployed for a new patient. In addition, because air does not normally flow through sensing tubes 138a and 138b, the presence of filter 405 in this arrangement does not significantly attenuate the pressure transfer or airflow to flowmeter 136.

In some embodiments, the housing of pressure sensor 134 is formed of a molded material such as plastic or any of various alternative polymer or composite materials, and filter 405 is molded to the housing. For example, the housing of filter 405 may be formed of the same material as the housing of pressure sensor 134, and they may be molded into a single piece. Alternatively, they can be formed of different materials and/or separate pieces that are bonded together using one of various available bonding materials. Moreover, filter 405 may be formed in a cavity or a dedicated orifice of pressure sensor 134. In addition, filter 405 may be connected or molded to portions of pressure sensor 134 other than its housing. For example, filter 405 may be connected to ports 140 and 145.

Although shown as a single unit in FIG. 4, filter 405 can also be implemented with more than one unit. For example, filter 405 may have separate filtering elements for ports 140 and 145, or the housing of filter 405 may comprise multiple components connected independently to different portions of the housing of pressure sensor 134.

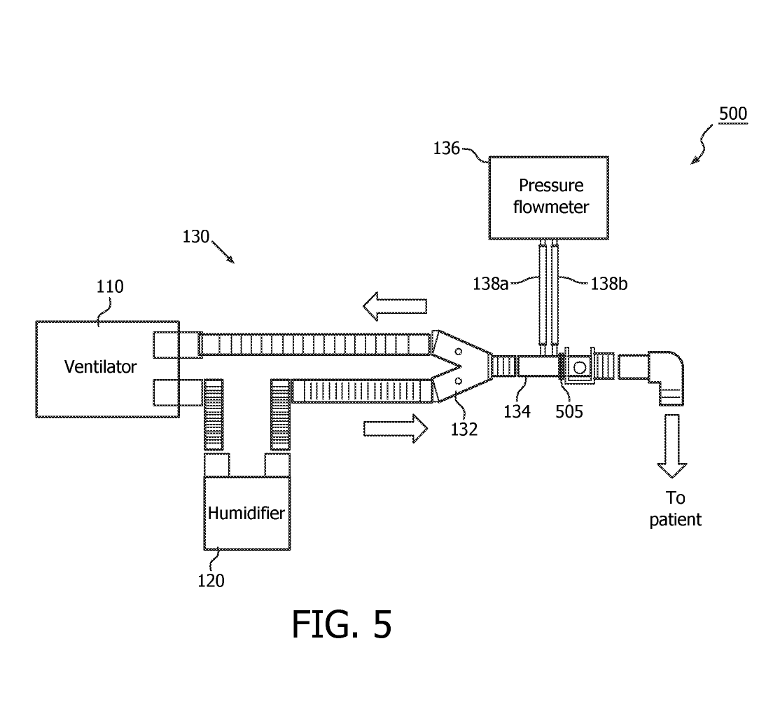

FIG. 5 is a functional block diagram of another ventilator system 500 comprising an in-line proximal differential pressure based flow sensor with an integrated filter according to a representative embodiment. Ventilator system 500 is the same as ventilator system 400 described above, except that a filter 505 is placed between a patient-side inlet of pressure sensor 134 and pressure sensing ports 140 and 145, and filter 405 is omitted. Like filter 405, filter 505 can be integrated with pressure sensor 134 in various ways, such as molding, bonding, and so forth.

As illustrated in FIG. 5, filter 505 is formed in-line with an inlet of pressure sensor 134. Accordingly, it may impede airflow through the housing of pressure sensor 134 more than filter 405. Nevertheless, this configuration may provide other potential benefits, such as convenient integration or enhanced control over the pressure differential between pressure sensing ports 140 and 145. These and other parameters, however, can be evaluated by designers or manufactures according to various considerations such as preference, empirical evaluations, and specific applications.

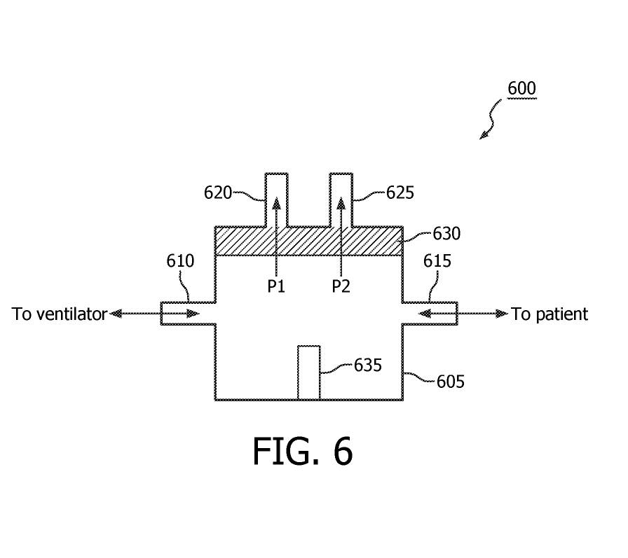

FIG. 6 is a conceptual block diagram illustrating an adapter 600 for a ventilator or respirator system according to a representative embodiment. Adapter 600 can be used, for example, in pressure sensor 134 as described above, or in a combination pressure sensor and gas sensor, such as a CO₂/O₂ sensor.

Because adapter 600 is presented in a conceptual form, it omits certain details that may be included in an actual implementation, and it does not necessarily reflect the actual dimensions, shape, and proportions of such an adapter as they may exist in a practical application. Nevertheless, such details may be determined or selected by those skilled in the art and having the benefit of this disclosure. In addition, although adapter 600 is shown with a substantially unitary housing, it can also be formed with multiple parts or stages. Moreover, various additional components can be included as part of adapter 600, such as an in-line gas sensor or an in-line filter.

In general, adapter 600 can be used in any arrangement for measurement of respiratory flow, respiratory gases, or both. For example, it can be used in combination with a ventilator or respirator for clinical or consumer applications. For explanation purposes, it will be assumed that adapter is designed for use in a medical context such that it can be connected between a patient and a respiratory apparatus.

Referring to FIG. 6, adapter 600 comprises a housing 605, first through fourth connectors 610, 615, 620 and 625, an integrated filter 630, and a resistive element 635. First connector 610 is configured for connection to a hose leading to a respirator, ventilator, or other respiratory apparatus. Connector 615 is configured for connection to a hose leading to a patient. Third and fourth connectors 620 and 625 are configured for sensing a pressure differential, i.e., they form part of a circuit for a differential pressure flow sensor. Pressure sensing tubes, although not shown, can be connected to third and fourth connectors 620 and 625 in order to transmit pressure and/or gas to a pressure flowmeter. Integrated filter 630 is configured to prevent patient originated infective agents from contaminating the pressure sensing tubes. Filter 630 typically comprises a bacterial and/or viral filter. Resistive element 635 creates an obstruction in the airflow through housing 605, which creates a pressure differential between first and second connectors 620 and 625, allowing a pressure based measurement of airflow.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

In the claims, as well as in the specification above, all transitional phrases such as “comprising,” “including,” “having,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An arrangement for measurement of respiratory flow or respiratory gases in an airway between a patient and a ventilator or respirator, the arrangement comprising:
an adapter comprising a housing having first through fourth openings;
a first connector attached to the adapter at the first opening and configured for connection to a first hose leading toward the ventilator or respirator;
a second connector attached to the adapter at the second opening and configured for connection to a second hose leading toward the patient;
third and fourth connectors attached to the adapter at the third and fourth openings and configured for connection with pressure sensing tubes to be used in conjunction with a differential pressure flow sensor located along a path between the first and second connectors; and
a filter integrated with the housing of the adapter and configured to prevent patient originated infective agents from contaminating the pressure sensing tubes.

2. The arrangement of claim 1, wherein the filter comprises a filter housing molded to the housing of the adapter.

3. The arrangement of claim 1, further comprising a pressure flowmeter connected to the pressure sensing tubes and configured to measure airflow through the adapter based on a pressure differential across the pressure sensing tubes.

4. The arrangement of claim 1, wherein the location of the filter is adjacent to the third and fourth connectors or adjacent to the first connector.

5. (canceled)

6. The arrangement of claim 1, wherein the adapter further comprises a resistive element adapted to create a pressure differential between the pressure sensing tubes.

7. A system, comprising:
a patient circuit configured to be connected between a ventilator and a patient;
a differential pressure sensor disposed in-line with the patient circuit and the patient and configured to sense a respiratory gas flow of the patient, the differential pressure sensor comprising a housing and first and second pressure sensing ports connected to the housing;
a flowmeter comprising first and second input ports in communication with the first and second pressure sensing ports of the differential pressure sensor, wherein the flowmeter is configured to measure a differential pressure between the first and second input ports and to output an electrical signal responsive to the differential pressure;
first and second pressure sensing tubes connected between the first and second pressure sensing ports of the differential pressure sensor and the first and second ports of the flowmeter; and
a contaminant blocking device integrated with the housing of the differential pressure sensor and configured to prevent contamination from being transmitted between the patient and the first and second pressure sensing ports and the first and second pressure sensing tubes.

8. The system of claim 7, wherein the patient circuit comprises a dual-limb patient circuit including a wye element having first and second ports connected to the dual limbs and having a third port connected to the differential pressure sensor.

9. The system of claim 7, wherein the contaminant blocking device comprises a filter housing connected to the housing of the differential pressure sensor, and a filter element mounted in the filter housing.

10. The system of claim 9, wherein the filter housing is molded to the housing of the differential pressure sensor.

11. (canceled)

12. The system of claim 7, wherein the contaminant blocking device is disposed adjacent to the pressure sensing tubes.

13. The system of claim 7, wherein the contaminant blocking device is disposed in-line with an airflow passing through the differential pressure sensor.

14. (canceled)

15. An apparatus, comprising:
a flow sensor configured to sense airflow between a respiration machine and a patient;
a first connector configured to communicate air between the flow sensor and the patient;
a second connector configured to communicate air between the flow sensor and the respiration machine;
a plurality of pressure sensing ports configured for connection to pressure sensing tubes and configured to communicate gas pressure between the flow sensor and a pressure flowmeter; and
a filter integrated with the flow sensor between the first connector and the pressure sensing ports and configured to communicate gas pressure therethrough while preventing contaminants from passing from the flow sensor to the pressure sensing ports and the pressure sensing tubes.

16. The apparatus of claim 15, wherein the filter is integrally formed with a housing of the flow sensor.

17. The apparatus of claim 16, wherein the filter comprises filter housing molded to a wall of the flow sensor.

18. The apparatus of claim 17, wherein the pressure sensing ports are located in the filter housing.

19. (canceled)

20. (canceled)
